(19) United States
(12) Patent Application Publication
(10) Pub. No.: US 2021/0326755 A1
(43) Pub. Date: Oct. 21, 2021

(54) LEARNING MODEL CREATION DEVICE, MATERIAL PROPERTY PREDICTION DEVICE, AND LEARNING MODEL CREATION METHOD AND PROGRAM

(71) Applicant: MITSUBISHI HEAVY INDUSTRIES, LTD., Tokyo (JP)

(72) Inventors: Keita HASHIMOTO, Tokyo (JP); Nobuyoshi KOMAI, Tokyo (JP); Takashi HONDA, Tokyo (JP); Nobuhiko SAITO, Tokyo (JP); Takahiro SHIRANE, Tokyo (JP); Ko ARISUE, Tokyo (JP); Yusuke YASHIRO, Tokyo (JP)

(21) Appl. No.: 17/217,296

(22) Filed: Mar. 30, 2021

(30) Foreign Application Priority Data
Apr. 20, 2020 (JP) 2020-074740

(51) Int. Cl. G06N 20/00; G06K 9/62; G06F 30/25

(52) U.S. Cl. CPC G06N 20/00; G06K 9/6228; G06K 9/6215; G06F 30/25; G06K 9/6262

(57) ABSTRACT

A learning model creation device includes, a verifying unit that verifies a learning model for predicting, from a predetermined manufacturing condition including at least one condition, a predetermined material property of a material manufactured under the manufacturing condition. The verifying unit includes, a first acquisition unit that acquires a first relationship that is a relationship between a selected condition, which is the condition selected from among the predetermined manufacturing condition, and the predetermined material property, the first relationship being obtained based on the learning model, a second acquisition unit that acquires a second relationship that is the relationship obtained based on material simulation, a similarity determination unit that determines presence or absence of similarity between the first relationship and the second relationship, and a relearning determination unit that determines a need for relearning of the learning model based on a determination result of the presence or absence of the similarity.

### CROSS-REFERENCE TO RELATED APPLICATIONS

This application claims the benefit of priority to Japanese Patent Application Number 2020-074740 filed on Apr. 20, 2020. The entire contents of the above-identified application are hereby incorporated by reference.

### TECHNICAL FIELD

The present disclosure relates to a prediction technique for a material property of a material such as a metal material.

### RELATED ART

For many metal materials used, for example, as structural members and functional members, manufacturing conditions such as a range of chemical composition and a range of heat treatment temperature and material properties required of materials (required properties) such as strength are specified by various standards such as JIS. However, even metal materials manufactured under the manufacturing conditions specified in this manner may not necessarily satisfy the required properties. Therefore, in order to prevent product incompatibility and deterioration in yield during manufacture, the manufacturing conditions are often managed in a stricter range within the specified range. While it is desired that the range of management of the manufacturing conditions (permissible range of variations) be defined through experimental verification, but more parameters that are managed as the manufacturing conditions (various conditions such as chemical composition and heat treatment temperature) are often more difficult from the perspective of cost and test time. Techniques for estimating, from manufacturing conditions, the properties of materials manufactured under the manufacturing conditions have also been proposed (see, e.g., JP 6086155 B, JP 2003-39180 A, and JP H05-287343 A).

Also, in recent years, material simulation technologies such as Integrated Computational Material Engineering (ICME) and techniques using machine learning such as Materials Informatics (MI) have been proposed, and it has been able to predict relationships between manufacturing conditions and material properties.

### SUMMARY

At present, however, ICME technology is not regarded as having sufficient accuracy for quantitative prediction of material properties. Also, it is difficult, in MI, to appraise whether the above relationships found by learning conform to a material science-based causal relationship. Thus, if these technologies are used alone to investigate the manufacturing conditions, there is a possibility that manufacturing conditions which deviate from the reality may be specified.

In light of the above circumstances, an object of at least one embodiment of the present invention is to provide a learning model creation device configured to create a learning model capable of highly reliable material property prediction.

The learning model creation device according to at least one embodiment of the present invention is a learning model creation device configured to create a learning model for predicting a material property of a material based on a manufacturing condition, the device including, a verifying unit configured to verify the learning model for predicting, from a predetermined manufacturing condition including at least one condition, a predetermined material property of a material manufactured under the manufacturing condition, in which the verifying unit includes a first acquisition unit configured to acquire a first relationship that is a relationship between a selected condition, which is the condition selected from among the predetermined manufacturing condition, and the predetermined material property, the first relationship being obtained based on the learning model, a second acquisition unit configured to acquire a second relationship that is the relationship obtained based on material simulation, a similarity determination unit configured to determine presence or absence of similarity between the first relationship and the second relationship, and a relearning determination unit configured to determine a need for relearning of the learning model based on a determination result of the presence or absence of the similarity.

The material property prediction device according to at least one embodiment of the present invention is a material property prediction device configured to predict a material property of a material, the device including, a prediction unit configured to predict, from a predetermined manufacturing condition, a predetermined material property of a material manufactured under the manufacturing condition, by using a learning model created by the learning model creation device described in any one of claims 1 to 5.

The learning model creation method according to at least one embodiment of the present invention is a learning model creation method for creating a learning model configured to predict a material property of a material based on a manufacturing condition, the method including, a verification step of verifying the learning model for predicting, from a predetermined manufacturing condition including at least one condition, a predetermined material property of a material manufactured under the manufacturing condition, in which the verification step includes, a step of acquiring a first relationship that is a relationship between a selected condition, which is the condition selected from among the predetermined manufacturing condition, and the predetermined material property, the first relationship being obtained based on the learning model, a step of acquiring a second relationship that is the relationship obtained based on material simulation, a step of determining presence or absence of similarity between the first relationship and the second relationship, and a step of determining a need for relearning of the learning model based on a determination result of the presence or absence of the similarity.

The learning model creation program according to at least one embodiment of the present invention is a program for creating a learning model configured to predict a material property of a material based on a manufacturing condition, the program being intended to cause a computer to, realize a verifying unit configured to verify the learning model for predicting, from a predetermined manufacturing condition including at least one condition, a predetermined material property of a material manufactured under the manufacturing condition, and realize, as the verifying unit, a first acquisition unit configured to acquire a first relationship that is a relationship between a selected condition, which is the condition selected from among the predetermined manufacturing condition, and the predetermined material property, the first relationship being obtained based on the learning model, a second acquisition unit configured to acquire a second relationship that is the relationship obtained based on material simulation, a similarity determination unit configured to determine presence or absence of similarity between the first relationship and the second relationship, and a relearning determination unit configured to determine a need for relearning of the learning model based on a determination result of the presence or absence of the similarity.

According to at least one embodiment of the present invention, a learning model creation device configured to create a learning model capable of highly reliable material property prediction is provided.

### BRIEF DESCRIPTION OF DRAWINGS

The disclosure will be described with reference to the accompanying drawings, wherein like numbers reference like elements.

FIG. 1 is a diagram schematically illustrating a configuration of a learning model creation device according to one embodiment of the present invention.

FIG. 2 is a diagram for exemplifying a first relationship according to one embodiment of the present invention.

FIG. 3A is a diagram for exemplifying a second relationship according to one embodiment of the present invention, illustrating a case where the second relationship is similar to the first relationship.

FIG. 3B is a diagram for exemplifying the second relationship according to one embodiment of the present invention, illustrating a case where the second relationship is not similar to the first relationship.

FIG. 4 is a diagram schematically illustrating a configuration of a material property prediction device according to one embodiment of the present invention.

FIG. 5 is a diagram illustrating a learning model creation method according to one embodiment of the present invention.

### DESCRIPTION OF EMBODIMENTS

Embodiments of the present invention will be described hereinafter with reference to the appended drawings. It is intended, however, that dimensions, materials, shapes, relative arrangements and the like of constituent elements illustrated in the embodiments are only examples and not intended to be limited to the scope of the present invention.

For instance, an expression of relative or absolute arrangement such as “in a direction”, “along a direction”, “parallel”, “orthogonal”, “centered”, “concentric” and “coaxial” shall not be construed as indicating only the arrangement in a strict literal sense, but also includes a state where the arrangement is relatively displaced by a tolerance, or by an angle or a distance within a range in which it is possible to achieve the same function.

For instance, an expression of an equal state such as “same”, “equal”, “uniform” and the like shall not be construed as indicating only the state in which the feature is strictly equal, but also includes a state in which there is a tolerance or a difference within a range where it is possible to achieve the same function.

Further, for instance, an expression of a shape such as a rectangular shape, a cylindrical shape or the like shall not be construed as only the geometrically strict shape, but also includes a shape with unevenness, chamfered corners or the like within the range in which the same effect can be achieved.

On the other hand, an expression such as “comprise”, “include”, “have”, “contain” and “constitute” are not intended to be exclusive of other constituent elements.

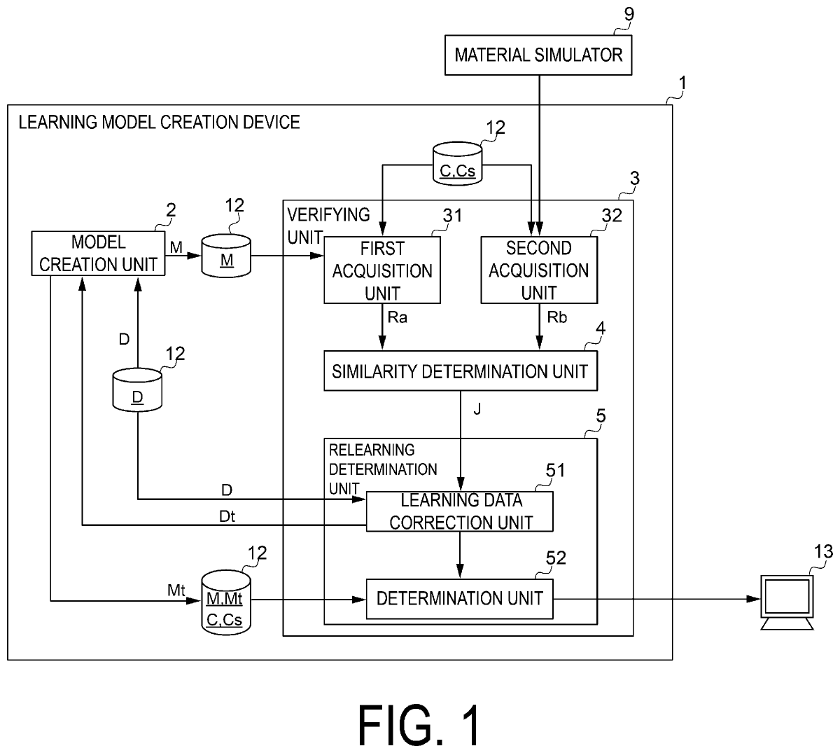

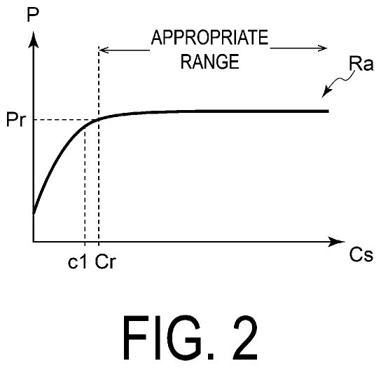

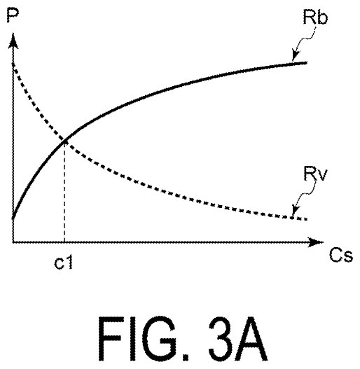

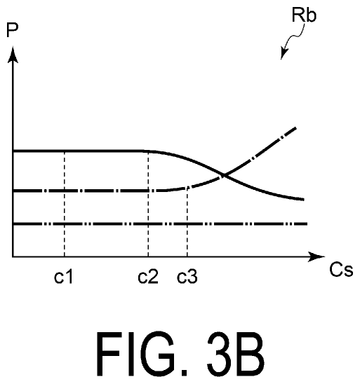

FIG. 1 is a diagram schematically illustrating a configuration of a learning model creation device 1 according to one embodiment of the present invention. FIG. 2 is a diagram for exemplifying a first relationship Ra according to one embodiment of the present invention. FIG. 3A is a diagram for exemplifying a second relationship Rb according to one embodiment of the present invention, illustrating a case where the second relationship Rb is similar to the first relationship Ra. FIG. 3B is a diagram for exemplifying the second relationship Rb according to one embodiment of the present invention, illustrating a case where the second relationship Rb is not similar to the first relationship Ra.

The learning model creation device 1 is a device configured to create a learning model M for predicting a desired material property (hereinafter, target material property P) of a desired material to be manufactured (hereinafter, referred to as target material) based on a predetermined manufacturing condition C used in the manufacture of the target material. The above-described target material may be, for example, a metal material used as a functional member or a structural member having a role of bearing a load. The above-described target material property P may be a mechanical property, such as tensile strength or creep rupture strength, required in accordance with an application of the target material such as a structural member. The manufacturing condition C described above includes at least one condition (management parameter) managed during manufacture of the target material, such as, for example, conditions for a range of the chemical composition of at least one chemical component forming the target material, and heat treatment conditions such as a range of heat treatment temperature.

Furthermore, the learning model M described above is created by learning (machine learning) a relationship between the predetermined manufacturing condition C described above and the target material property P of the target material manufactured under the manufacturing condition C. This learning may be performed on learning data D (training data) by a well-known machine learning technique. The learning data D may be configured by a plurality of data in which a measurement result of the target material property P obtained, for example, by measuring the target material manufactured in the past and the manufacturing condition C used in the manufacture of the target material from which the measurement results have been obtained are correlated (associated) with each other. Alternatively, the learning data D may be configured by a plurality of data in which the target material property P obtained, for example, from a literature or numerical analysis (material simulation) and the manufacturing condition C are correlated (associated) with each other. These data may be combined. The learning model M created in this manner outputs, with respect to the input manufacturing condition C, a predicted value of the target material property P of the target material manufactured under the manufacturing condition C.

Then, as illustrated in FIG. 1, the learning model creation device 1 includes a verifying unit 3 configured to verify the learning model M described above. In the embodiment illustrated in FIG. 1, the learning model creation device 1 further includes a model creation unit 2 configured to create the learning model M through performing machine learning of the learning data D. The verifying unit 3 acquires the learning model M created by the model creation unit 2, and verifies the learning model M in a manner as described below.

Specifically, as illustrated in FIG. 1, the above-described verifying unit 3 includes a first acquisition unit 31, a second acquisition unit 32, a similarity determination unit 4, and a relearning determination unit 5. Each of these functional units will be described below. Note that, in the following description, a case will be taken as an example where the target material is a metal material, the manufacturing condition C includes at least one of a range of chemical composition or heat treatment conditions that define a range of heat treatment temperature and the like, and the target material property P is creep rupture strength.

Note that the learning model creation device 1 is configured by a computer. Note that this computer includes a processor such as a CPU (not illustrated) and a storage unit 12 such as a memory, i.e., a ROM or a RAM. The storage unit 12 may include an external storage unit. The processor performs operation (such as computation of data) according to a command of a program (learning model creation program, material property prediction program) loaded into a memory (main storage unit), and thus each of the above-described functional units is realized. To rephrase, the above-described program is software by which the computer realizes each of the functional units, which will be described later, and the program may be stored in a computer-readable storage medium.

The first acquisition unit 31 is a functional unit configured to acquire a first relationship Ra (see FIG. 2) that is a relationship between a selected condition Cs, which is a condition selected from among the predetermined manufacturing conditions C, and the target material property P, the relationship being obtained based on the learning model M described above. The second acquisition unit 32 is a functional unit configured to acquire a second relationship Rb (see FIGS. 3A and 3B) that is a relationship between the same selected condition Cs as described above and the target material property P, the relationship being obtained based on the material simulation of the material. That is, the same (same type of) relationships (Ra and Rb) obtained by using mutually different techniques, i.e., the learning model M and the material simulation, are acquired by the first acquisition unit 31 and the second acquisition unit 32, respectively.

More specifically, as illustrated in FIGS. 2 to 3B, the above-described relationships (Ra and Rb) indicate a change in the target material property P (creep rupture strength) depending on a change in the selected condition Cs. The above-described selected condition Cs is preferably selected such that the change in the value of the target material property P output from the learning model M is relatively large with respect to the change in the selected condition Cs. This is because, when there is a variation in condition having a great influence (sensitivity) on the output of the learning model M, the target material property P greatly changes in accordance with the variation, and relatively more stringent condition setting is required than in the case of a condition whose influence as described above is relatively small. Note that, since a condition whose influence is small, as described above, has a wide permissible variation range, management may be less stringent such that it is sufficient when the condition falls within a range as specified by various standards such as JIS.

In general, in the learning model M, when the learning data D in which the manufacturing condition C and the target material property P are associated with each other is learned, it is possible to directly obtain the target material property P from the desired manufacturing condition C. However, in the material simulation, it may not be possible to directly obtain the target material property P. For example, at present, the creep rupture strength cannot be directly calculated in the material simulation. In such a case, the material simulation may determine a particle size of a precipitate of the target material, for example, having a correlation with the creep rupture strength, and calculate the creep rupture strength on the basis of the determined particle size. The creep rupture strength is known to decrease as the above-described particle size increases, and to increase as the above-described particle size decreases. In other words, the second relationship Rb may be determined by determining a physical property value correlated with the target material property P, which can be directly calculated by the material simulation (see a dashed line in FIG. 3A), and calculating the target material property P on the basis of the determined physical property value.

The similarity determination unit 4 is a functional unit configured to determine presence or absence of similarity between the above-described first relationship Ra and the above-described second relationship Rb. Specifically, a tendency of the change in the target material property P with respect to the change in the selected condition Cs in the first relationship Ra is compared with a tendency of the change in the target material property P with respect to the change in the selected condition Cs in the second relationship Rb. The presence or absence of the similarity described above may also be determined based on the comparison between these tendencies. That is, when the tendencies are similar, it is determined that the similarity is present, and when the tendencies are different, it is determined that the similarity is absent.

The presence or absence of the similarity will be described using the exemplifications in FIGS. 2 to 3B.

For example, in the exemplification in FIG. 2, as for the first relationship Ra obtained by the learning model M, the target material property P increases relatively sharply with the increase of the value in the selected condition Cs until the value of the selected condition Cs reaches around c1, and thereafter increases relatively gently. In other words, the rate of increase in the target material property P when the value of the selected condition Cs is not smaller than around c1 is smaller than the rate of increase in the target material property P when it is not greater than around c1.

In contrast, when the second relationship Rb obtained by the material simulation is as illustrated by the solid line in FIG. 3A, for example, it is determined that the similarity is present between the first relationship Ra and the second relationship Rb in the embodiment illustrated in FIG. 1. As for the second relationship Rb illustrated in FIG. 3A, the target material property P increases relatively sharply with the increase of the value in the selected condition Cs until the value of the selected condition Cs reaches around c1, and thereafter increases relatively gently. In other words, the rate of increase in the target material property P when the value of the selected condition Cs is not smaller than around c1 is smaller than the rate of increase in the target material property P when it is not greater than around c1, which is similar to that for the first relationship Ra illustrated in FIG. 2. Thus, it is determined that the similarity is present between the first relationship Ra and the second relationship Rb.

Note that a dotted line illustrated in FIG. 3A illustrates a relationship (third relationship Rv) between the selected condition Cs and a physical property value correlated with the target material property P, as an example of the particle size with respect to the creep rupture strength described above. By converting this dotted line, a solid line is obtained. Furthermore, the selected condition Cs may be a content of a prescribed chemical component (for example, carbon (C), manganese (Mn), or the like), or a heat treatment temperature.

On the other hand, when the second relationship Rb obtained by the material simulation is as illustrated in FIG. 3B, for example, it is determined that the similarity is absent between the first relationship Ra and the second relationship Rb in the embodiment illustrated in FIG. 1. FIG. 3B exemplifies three types of second relationships Rb (solid line, long dashed short dashed line, and long dashed double-short dashed line). In any example, the second relationship Rb differs from the first relationship Ra in that the value of the target material property P obtained by the material simulation is generally constant, for example, until the value of the selected condition Cs reaches around c1. The second relationship Rb differs, in general tendency, from the first relationship Ra. Furthermore, a tendency of the solid line in FIG. 3B differs, for example, in that the value of the target material property P is generally constant or slightly increases until the value of the selected condition Cs reaches around c2 having a value more than the above-described c1, and tends to decrease when it is not smaller than around c2. A tendency of the long dashed short dashed line in FIG. 3B differs in that the value of the target material property P is generally constant or slightly increases until the value of the selected condition Cs reaches around c3 having a value more than the above-described c1 and c2, and comes to show an increase tendency when it reaches around c3, and in terms of the form of the increase. A tendency of the long dashed double-short dashed line differs, for example, in that the value of the target material property P is generally constant regardless of the value of the selected condition Cs. From the perspectives, it can be said that the second relationship Rb illustrated in FIG. 3B differs, in general tendency, from the first relationship Ra.

The relearning determination unit 5 is a functional unit configured to determine a need for relearning of the learning model M on the basis of a determination result J of the presence or absence of the above-described similarity. Specifically, in a case where, for example, the determination result J is obtained that the similarity is present, each predicted value of the target material property P output from the learning model M when the selected condition Cs is gradually changed in any manufacturing condition C, and the tendency of the change thereof will be supported by the material simulation performed in accordance with the material science. In other words, such a learning model M can be said to perform reliable prediction from the perspective of the material science. Thus, the use of such a reliable learning model M makes it possible to decide the set value of the selected condition Cs that satisfies the required value of the target material property P.

For example, in a case where the reliability of the learning model M is confirmed based on the first relationship Ra illustrated in FIG. 2, assuming that the required value (the required property Pr) of the target material property P is Pr or greater, the target material property P is Pr or greater when the value of the selected condition Cs is Cr or greater in the exemplification in FIG. 2. So, it is sufficient that the selected condition Cs be set to Cr or greater.

Conversely, when the determination result J is obtained that the similarity is absent, the output of the learning model M will not be supported by the material simulation, so the results of prediction by the learning model M are not reliable. In such a case, it may be unconditionally determined that relearning of the learning model M is necessary, or may be determined that relearning is necessary when a predetermined condition is satisfied, as described below.

In the embodiment illustrated in FIG. 1, the model creation unit 2 creates (temporarily creates) the learning model M, which is unverified, through machine learning of the learning data D stored in the storage unit 12 in advance, and stores the created learning model M in the storage unit 12. Then, the first acquisition unit 31 generates a plurality of set values for the selected condition Cs in the manufacturing condition C specified by the user or the like, and acquires the output value of the target material property P by inputting the manufacturing condition C in the learning model M while changing the selected condition Cs in the manufacturing condition C to each of the plurality of set values in turn, thereby acquiring the first relationship Ra. The second acquisition unit 32 acquires the second relationship Rb by acquiring each of the values of the target material property P corresponding to the plurality of set values of the selected condition Cs in the same manufacturing condition C, with using the material simulator 9 that is capable of executing the material simulation. The first acquisition unit 31 and the second acquisition unit 32 are respectively connected to the similarity determination unit 4, and input the first relationship Ra and the second relationship Rb into the similarity determination unit 4.

The similarity determination unit 4 is connected to the relearning determination unit 5, and determines the presence or absence of the similarity between the first relationship Ra and the second relationship Rb when they are input, and inputs the determination result J into the relearning determination unit 5. Then, the relearning determination unit 5 determines the need for relearning by executing processing in accordance with the input determination result J. As illustrated in FIG. 1, the relearning determination unit 5 may be connected to an output device, such as a display 13, which can notify a user of the determination result J of the need for relearning, and may output the determination result J to the output device. In the case where it is determined that the relearning is necessary, for example, the learning data D may be further improved, and then relearning by the model creation unit 2 may be performed in accordance with a user instruction or the like.

According to the above-described configuration, the validity of the learning model M, in which the relationship between the manufacturing condition C and the target material property P for the target material has been learned is verified on the basis of a simulation result from the material simulation serving as numerical analysis based on the material science. Specifically, the relationship (first relationship Ra, second relationship Rb) between any condition (selected condition Cs) included in the manufacturing condition C and the target material property P is determined on the basis of each of the learning model M and the material simulation, and the validity of the learning model M is verified on the basis of the determination result J of the similarity between the two relationships.

In other words, the relationship (causal relationship) between the manufacturing condition C obtained based on the material science and the target material property P is used to support the validity of the learning model M, which generally has poor scientific basis. This makes it possible to create a highly reliable learning model M. Additionally, the learning model M whose validity has been verified is used to predict the target material property P of the target material manufactured under any manufacturing condition C, and thus a highly accurate prediction result can be obtained. Thus, the manufacturing condition C for making the target material property P of the target material satisfy the required quality is decided using the learning model M, and the target material is manufactured under the decided manufacturing condition C, thereby making it possible to more reliably acquire the target material satisfying the required quality.

Next, some embodiments related to the above-described relearning determination unit 5 will be described.

One possible cause of the case where the determination result J is obtained that the above-described similarity is absent is insufficient learning at the time of creation of the learning model M (insufficient learning data D). However, when relearning is unconditionally performed on the determination result J that the above-described similarity is absent, the influence of the selected condition Cs on the target material property P is small, and relearning may be forcibly required even when it is sufficient to manage the condition within a range as specified by various standards. Thus, in order to avoid such an inconvenience, in some embodiments, in a case where the manufacturing condition C includes a plurality of conditions, the relearning determination unit 5 may determine that the relearning is necessary when a predetermined condition is satisfied in a case where the similarity determination unit 4 determines that the above-described similarity is absent.

Specifically, in some embodiments, the relearning determination unit 5 includes a learning data correction unit 51 configured to generate learning data with the selected condition Cs excluded from learning data D used during learning of the learning model M (hereinafter, corrected learning data Dt) when the above-described similarity determination unit 4 determines that the above-described similarity is absent, and a determination unit 52 configured to determine that relearning of the learning model M is necessary when a difference between predicted values of the target material property P that are respectively obtained based on corrected learning model Mt obtained by learning the generated corrected learning data Dt and the learning model M that is under verification is greater than or equal to a predetermined threshold value. The difference between the predicted values of the learning model M and the corrected learning model Mt may be obtained by determining the first relationships Ra of the respective models and comparing these first relationships Ra with each other.

In other words, when there is no significant change between the predicted values of the target material property P for any manufacturing condition C, respectively obtained by the learning model M that has learned the learning data D including the selected condition Cs and the corrected learning model Mt that has learned the corrected learning data Dt with the selected condition Cs excluded, it can be appraised that the influence of the selected condition Cs on the target material property P is sufficiently small. In a case where the influence of the selected condition Cs on the target material property P is sufficiently small in this manner, the need for strict management of the selected condition Cs is poor, and it is possible to appraise that the selected condition Cs need not be narrowed.

Conversely, in a case where the influence of the selected condition Cs on the target material property P is too great to ignore, the possibility of manufacturing a target material that fails to satisfy the quality requirement for the target material property P is high, if the selected condition Cs is not strictly managed. This leads to product incompatibility and deterioration in yield during manufacture. So, it is determined that relearning is necessary, to utilize the learning model M for the purpose of finding a set value which is not excessively strict. In this case, the learning data is additionally collected and then relearning is executed.

In the embodiment illustrated in FIG. 1, the learning data correction unit 51 generates the corrected learning data Dt by acquiring the learning data D used to create the learning model M that is under verification, and deleting the selected condition Cs in each of the plurality of data (records) constituting the acquired learning data D. The corrected learning model Mt is created by the above-described model creation unit 2 with machine learning the corrected learning data Dt generated by the learning data correction unit 51. Then, the determination unit 52 determines the need for relearning of the learning model M that is under verification, by determining the predicted value of the target material property P for the manufacturing condition C using each of the learning model M that is under verification and the corrected learning models Mt.

According to the above-described configuration, when the difference between the prediction result of the target material property P by each of the learning model M created by learning the learning data D including the selected condition Cs and the corrected learning model Mt created by learning the corrected learning data Dt with the selected condition Cs excluded is too great to ignore, it is determined that relearning is necessary.

That is, if a difference between the outputs of the learning model M and the corrected learning model Mt described above is too great to ignore, the selected condition Cs will have a great influence on the prediction. Nevertheless, it is determined that the similarity is absent between the first relationship Ra and the second relationship Rb, and thus insufficient learning is conceivable. Thus, in this case, it is determined that relearning of the learning model M is necessary. Conversely, when there is no great difference between the two outputs, the influence of the selected condition Cs on the prediction will be small. Thus, it can be considered that the selected condition Cs just has to conform to various standards, and it is possible to avoid the cost increase or the like caused by strictly managing the condition having a small influence on the target material property P.

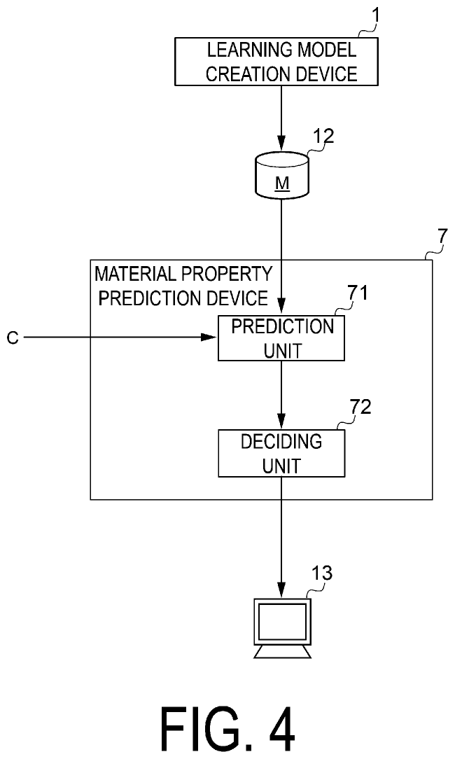

Next, a material property prediction device 7 that performs prediction using the learning model M described above will be described using FIG. 4. FIG. 4 is a diagram schematically illustrating a configuration of the material property prediction device 7 according to one embodiment of the present invention.

The material property prediction device 7 is a device for predicting the target material property P of the target material. As illustrated in FIG. 4, the material property prediction device 7 includes a prediction unit 71 configured to predict, from a predetermined manufacturing condition C, the target material property P of the target material manufactured under this manufacturing condition using the learning model M created (verified) by the learning model creation device 1 described above.

In the embodiment illustrated in FIG. 4, the prediction unit 71 inputs the manufacturing condition C into the learning model M, for example, when acquiring the predetermined manufacturing condition C, for example, through input of the manufacturing condition C from a user or the like. Then, the prediction unit 71 acquires the output value of the learning model M as a result of the input, and outputs the acquired output value of the learning model M as a predicted value of the target material property P to a deciding unit 72, which will be described later. Note that the prediction unit 71 may output the predicted value of the target material property P to an output device such as the display 13.

According to the above-described configuration, the target material property P manufactured under any manufacturing condition C is predicted using the learning model M created by the learning model creation device 1 and verified by the verifying unit 3. Thus, a highly reliable prediction result of the target material property P can be obtained.

Additionally, in some embodiments, as illustrated in FIG. 4, the material property prediction device 7 may further include the deciding unit 72 configured to decide recommended setting which is recommended for the selected condition Cs on the basis of a result of prediction by the above-described prediction unit 71. Specifically, the deciding unit 72 may decide a value within the appropriate range, for example, the loosest value within the appropriate range in which the requirement for the target material property P of the target material is satisfied (see FIG. 2), as recommended setting of the selected condition Cs, based on the first relationship Ra.

For example, in the example illustrated in FIG. 2, in a case where Cr, which is a boundary between an appropriate range and the other range of the selected condition Cs, is a set value that is most loose for the selected condition Cs, the recommended setting for the selected condition Cs may be Cr.

According to the above-described configuration, the recommended setting of the selected condition Cs is decided on the basis of the prediction result obtained using the verified learning model M created by the learning model creation device 1. This makes it possible to more reliably acquire the target material satisfying the required quality.

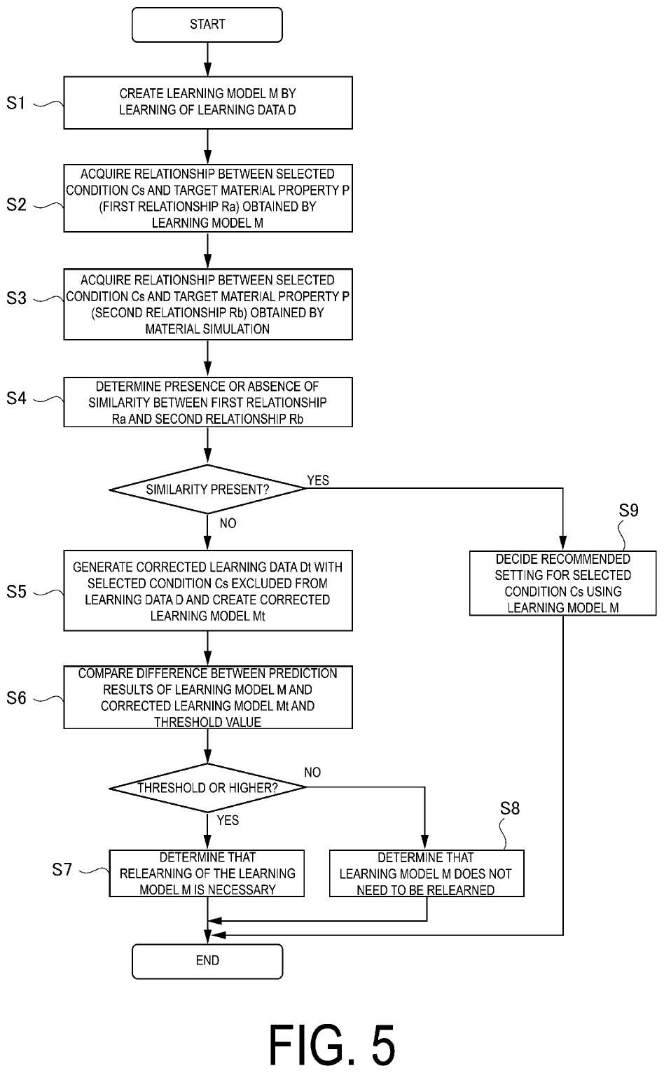

A learning model creation method corresponding to the processing of the learning model creation device 1 described above will be described below using FIG. 5. FIG. 5 is a diagram illustrating a learning model creation method according to one embodiment of the present invention.

The learning model creation method (material property prediction method) is a method for creating the learning model M described above. As illustrated in FIG. 5, the learning model creation method includes a verification step (S2) of verifying the learning model M described above. In the embodiment illustrated in FIG. 5, the learning model creation method further includes a model creation step (S1) of creating the above-described learning model M by learning the learning data D, and the verification step verifies the learning model M created by the model creation step.

The verification step includes a first acquisition step of acquiring the first relationship Ra described above, a second acquisition step of acquiring the second relationship Rb described above, a similarity determination step of determining presence or absence of similarity between the first relationship Ra and the second relationship Rb obtained through the first acquisition step and the second acquisition step, and a relearning determination step of determining the need for relearning of the learning model M based on the determination result J of the presence or absence of the similarity through the similarity determination step.

Also, the above-described relearning determination step may include a learning data correction step of generating the above-described corrected learning data Dt in a case where it is determined that the similarity is absent through the similarity determination step, and a determination step of determining that relearning of the learning model M is necessary in a case where the difference between the predicted values of the target material property P respectively obtained on the basis of each of corrected learning model Mt obtained by learning the generated corrected learning data Dt and the learning model M is greater than or equal to the predetermined threshold value.

The model creation step and verification step are similar to the contents of the processing executed by the model creation unit 2 and the verifying unit 3, respectively, described above, and thus detailed description thereof will be omitted.

Additionally, in some embodiments, there may be provided a prediction step of predicting, from the predetermined manufacturing condition, the target material property P of the target material manufactured under the manufacturing condition using the learning model M created by the learning model creation method described above (material property prediction method). Furthermore, in some embodiments, the material property prediction method may further include a decision step of deciding recommended setting for the selected condition Cs on the basis of the prediction result through the prediction step.

The embodiment illustrated in FIG. 5 will be described. In step S1, a learning model M before verification (unverified) is created (temporarily created) by learning the learning data D. In step S2, a first relationship Ra obtained by learning model M created in step S1 is acquired. In step S3, a second relationship Rb obtained by material simulation using a material simulator 9 or the like is acquired. Then, in step S4, the presence or absence of similarity between the first relationship Ra and the second relationship Rb obtained in these above described steps, respectively, is determined.

When the determination result J in step S4 presents that the similarity is absent, the above-described corrected learning data Dt is generated in step S5, and the above-described corrected learning model Mt is creased by learning the corrected learning data Dt. In step S6, each of the learning model M created in step S1 and the corrected learning model Mt created in step S5 is used to determine the prediction result (output) for the manufacturing condition C, and the difference between these prediction results and a predetermined threshold value are compared with each other. As a result, when the difference between the prediction results is greater than or equal to the threshold value, it is determined that relearning of the learning model M is necessary in step S7. Conversely, when the difference between the prediction results is smaller than the threshold value, it can be appraised that any value within the range of the selected condition Cs defined in various standards may be employed. Therefore, in step S8, it is determined that the relearning of the learning model M is unnecessary.

Conversely, when the determination result J in step S4 presents that the similarity is present, the verification of the prediction of the target material property P by the learning model M created in step Si has been completed. In the present embodiment, in this case, in step S9, the learning model M which has been verified is used to decide the recommended setting for the selected condition Cs.

Note that the order of steps S2 and S3 may be reversed, or these steps may be performed in parallel. In addition, the flow is terminated after executing any of steps S7 to S9 in FIG. 5, but, in a case where a plurality of conditions are included in the manufacturing condition C, steps S2 to S9 may be repeated. In other words, in a case where verification is performed through steps S2 to S9 on the other conditions that are not yet verified, the step S2 and the subsequent steps may be repeatedly executed with selecting one of the other conditions to use as a new selected condition Cs until the condition to be set as the selected condition Cs is not left.

The present invention is not limited to the embodiments described above, and also includes modification aspects of the above-described embodiments as well as appropriate combinations of these aspects.

### Notes

(1) The learning model creation device (1) according to at least one embodiment of the present invention is a learning model creation device (1) configured to create a learning model (M) for predicting a material property of a material based on a manufacturing condition (C), the device (1) including, a verifying unit (3) configured to verify the learning model (M) for predicting, from a predetermined manufacturing condition (C) including at least one condition, a predetermined material property of a material manufactured under the manufacturing condition (C), wherein the verifying unit (3) includes, a first acquisition unit (31) configured to acquire a first relationship (Ra) that is a relationship between a selected condition (Cs), which is the condition selected from among the predetermined manufacturing condition (C), and the predetermined material property, the first relationship being obtained based on the learning model (M), a second acquisition unit (32) configured to acquire a second relationship (Rb) that is the relationship obtained based on material simulation, a similarity determination unit (4) configured to determine presence or absence of similarity between the first relationship (Ra) and the second relationship (Rb), and a relearning determination unit (5) configured to determine a need for relearning of the learning model (M) based on a determination result (J) of the presence or absence of the similarity.

According to the configuration described in the above-described (1), the validity of the learning model (M) that has learned the relationship between the manufacturing condition (C) including conditions such as composition and heat treatment temperature and a predetermined material property (hereinafter, target material property (P)) such as a mechanical property, e.g., tensile strength, for a material to be manufactured (hereinafter, target material) such as a metal material or a functional material is verified on the basis of a simulation result from the material simulation serving as numerical analysis based on the material science. Specifically, the relationship (first relationship (Ra), second relationship (Rb)) between any condition (selected condition (Cs)) included in the manufacturing condition (C) and the target material property (P), for example, in terms of the tendency of the change in the target material property (P) in accordance with the change in the setting (set value) of the selected condition (Cs), is determined on the basis of each of the learning model (M) and the material simulation, and the validity of the learning model (M) is verified on the basis of the determination result (J) of the similarity between the two relationships.

In other words, the relationship (causal relationship) between the manufacturing condition (C) obtained based on the material science and the target material property (P) is used to support the validity of the learning model (M), which generally has poor scientific basis. This makes it possible to create a highly reliable learning model (M). Additionally, the learning model (M) whose validity has been verified is used to predict the target material property (P) of the target material manufactured under any manufacturing condition (C), and thus a highly accurate prediction result can be obtained. Thus, the manufacturing condition (C) for making the target material property (P) of the target material satisfy the required quality is determined using the learning model (M), and the target material is manufactured under the decided manufacturing condition (C), thereby making it possible to more reliably acquire the target material satisfying the required quality.

(2) In some embodiments, in the configuration described in the above-described (1), the manufacturing condition (C) includes a plurality of the conditions, and the relearning determination unit (5) includes, a learning data correction unit (51) configured to generate corrected learning data (Dt) with the selected condition (Cs) excluded from learning data (D) used during learning of the learning model (M) when it is determined that the similarity is absent, and a determination unit (52) configured to determine that relearning of the learning model (M) is necessary when a difference between predicted values of the predetermined material property that are respectively obtained based on corrected learning model (Mt) obtained by learning the corrected learning data (Dt) and the learning model (M) is greater than or equal to a predetermined threshold value.

According to the configuration described in the above-described (2), when the difference between the prediction result of the target material property (P) by each of the learning model (M) created by learning the learning data (D) including the selected condition (Cs) and the learning model (corrected learning model (Mt)) created by learning the learning data (corrected learning data Dt) with the selected condition (Cs) excluded is too great to ignore, it is determined that relearning is necessary.

That is, if a difference between the outputs of the learning model (M) and the corrected learning model (Mt) described above is too great to ignore, the selected condition (Cs) will have a great influence on the prediction. Nevertheless, it is determined that the similarity is absent between the first relationship (Ra) and the second relationship (Rb), and thus insufficient learning is conceivable. Thus, in this case, it is determined that relearning of the learning model (M) is necessary. Conversely, when there is no great difference between the two outputs, the influence of the selected condition (Cs) on the prediction will be small. Thus, it can be considered that the selected condition (Cs) just has to conform to various standards, and it is possible to avoid the cost increase or the like caused by strictly managing the condition having a small influence on the target material property (P).

(3) In some embodiments, in the configurations described in the above-described (1) and (2), the similarity determination unit (4) is configured to determine the presence or absence of the similarity based on comparison of tendency of a change in the predetermined material property in accordance with the change in the selected condition (Cs), in each of the first relationship (Ra) and the second relationship (Rb).

According to the configuration described in the above-described (3), the tendency of a change in the target material property (P) in accordance with the change in the selected condition (Cs) is obtained for each of the first relationship (Ra) and the second relationship (Rb), and the presence or absence of the similarity described above is determined based on the change in the obtained tendency. This makes it possible to appropriately determine the presence or absence of the similarity described above.

(4) In some embodiments, in the configurations described in the above-described (1) to (3), the second relationship (Rb) is determined based on a physical property value correlated with the predetermined material property, where the physical property value can be calculated by the material simulation.

According to the configuration described in the above-described (4), it may not be possible to directly calculate the target material property (P) in the material simulation. In such a case, a physical property value correlated with the target material property (P) is determined, and the second relationship (Rb) is determined based on the correlation (third relationship (Rv)) between the physical property value and the target material property (P). This allows verification of the validity of the learning model (M) based on the simulation result from the material simulation.

(5) In some embodiments, in the configurations described in the above-described (1) to (4), the material is a metal material.

According to the configuration described in the above-described (5), a material property of the metal material is predicted. As a result, it is possible to improve the reliability of the learning model (M) for predicting the target material property (P) of the metal material from the manufacturing condition (C).

(6) A material property prediction device (7) according to at least one embodiment of the present invention is a material property prediction device (7) configured to predict a material property of a material, the device including, a prediction unit (71) configured to predict, from a predetermined manufacturing condition (C), a predetermined material property of a material manufactured under the manufacturing condition (C), by using a learning model (M) created by the learning model creation device (1) described in any one of (1) to (5) above.

According to the configuration of (6) above, the target material property (P) manufactured under any manufacturing condition (C) is predicted using the learning model (M) created by the learning model creation device (1) and verified by the verifying unit (3). Thus, a highly reliable prediction result of the target material property (P) can be obtained.

(7) In some embodiments, the configuration described in the above-described (6) further includes a deciding unit (72) configured to decide recommended setting recommended for the selected condition (Cs) based on a result of prediction by the prediction unit (71).

According to the configuration described in the above-described (7), the recommended setting of the selected condition (Cs) is decided based on the prediction result obtained using the verified learning model (M) created by the learning model creation device (1). This makes it possible to more reliably acquire the target material satisfying the required quality.

(8) The learning model creation method according to at least one embodiment of the present invention is a learning model creation method for creating a learning model (M) configured to predict a material property of a material based on a manufacturing condition (C), the method including, a verification step of verifying the learning model (M) for predicting, from a predetermined manufacturing condition (C) including at least one condition, a predetermined material property of a material manufactured under the manufacturing condition (C), where the verification step includes, a step of acquiring a first relationship (Ra) that is a relationship between a selected condition (Cs), which is the condition selected from among the predetermined manufacturing condition (C), and the predetermined material property, the first relationship (Ra) being obtained based on the learning model (M), a step of acquiring a second relationship (Rb) that is the relationship obtained based on material simulation, a step of determining presence or absence of similarity between the first relationship (Ra) and the second relationship (Rb), and a step of determining a need for relearning of the learning model (M) based on a determination result (J) of the presence or absence of the similarity.

According to the configuration described in the above-described (8), effects similar to those described in the above-described (1) are exhibited.

(9) The learning model creation program according to at least one embodiment of the present invention is a learning model creation program for creating a learning model (M) configured to predict a material property of a material based on a manufacturing condition (C), the program being intended to cause a computer to, realize a verifying unit (3) configured to verify the learning model (M) for predicting, from a predetermined manufacturing condition (C) including at least one condition, a predetermined material property of a material manufactured under the manufacturing condition (C), and realize, as the verifying unit (3), a first acquisition unit (31) configured to acquire a first relationship (Ra) that is a relationship between a selected condition (Cs), which is the condition selected from among the predetermined manufacturing condition (C), and the predetermined material property, the first relationship being obtained based on the learning model (M), a second acquisition unit (32) configured to acquire a second relationship (Rb) that is the relationship obtained based on material simulation, a similarity determination unit (4) configured to determine presence or absence of similarity between the first relationship (Ra) and the second relationship (Rb), and a relearning determination unit (5) configured to determine a need for relearning of the learning model (M) based on a determination result (J) of the presence or absence of the similarity.

According to the configuration described in the above-described (9), effects similar to those described in the above-described (1) are exhibited.

While preferred embodiments of the invention have been described as above, it is to be understood that variations and modifications will be apparent to those skilled in the art without departing from the scope and spirit of the invention. The scope of the invention, therefore, is to be determined solely by the following claims.

1. A learning model creation device configured to create a learning model for predicting a material property of a material based on a manufacturing condition, the device comprising:
a verifying unit configured to verify the learning model for predicting, from a predetermined manufacturing condition including at least one condition, a predetermined material property of a material manufactured under the manufacturing condition, wherein
the verifying unit includes,
a first acquisition unit configured to acquire a first relationship that is a relationship between a selected condition, which is the condition selected from among the predetermined manufacturing condition, and the predetermined material property, the first relationship being obtained based on the learning model,
a second acquisition unit configured to acquire a second relationship that is the relationship obtained based on material simulation,
a similarity determination unit configured to determine presence or absence of similarity between the first relationship and the second relationship, and
a relearning determination unit configured to determine a need for relearning of the learning model based on a determination result of the presence or absence of the similarity.

2. The learning model creation device according to claim 1, wherein
the manufacturing condition includes a plurality of the conditions, and
the relearning determination unit includes,
a learning data correction unit configured to generate corrected learning data with the selected condition excluded from learning data used during learning of the learning model when it is determined that the similarity is absent, and
a determination unit configured to determine that relearning of the learning model is necessary when a difference between predicted values of the predetermined material property that are respectively obtained based on corrected learning model obtained by learning the corrected learning data and the learning model is greater than or equal to a predetermined threshold value.

3. The learning model creation device according to claim 1, wherein the similarity determination unit is configured to determine the presence or absence of the similarity based on comparison of tendency of a change in the predetermined material property in accordance with the change in the selected condition, in each of the first relationship and the second relationship.

4. The learning model creation device according to claim 1, wherein the second relationship is obtained based on a physical property value correlated with the predetermined material property, where the physical property value can be calculated by the material simulation.

5. The learning model creation device according to claim 1, wherein the material is a metal material.

6. A material property prediction device configured to predict a material property of a material, the device comprising:
a prediction unit configured to predict, from a predetermined manufacturing condition, a predetermined material property of a material manufactured under the manufacturing condition, by using a learning model created by the learning model creation device described in claim 1.

7. The material property prediction device according to claim 6, further comprising a deciding unit configured to decide recommended setting recommended for the selected condition based on a result of prediction by the prediction unit.

8. A learning model creation method for creating a learning model configured to predict a material property of a material based on a manufacturing condition, the method comprising:
a verification step of verifying the learning model for predicting, from a predetermined manufacturing condition including at least one condition, a predetermined material property of a material manufactured under the manufacturing condition, wherein
the verification step includes,
a step of acquiring a first relationship that is a relationship between a selected condition, which is the condition selected from among the predetermined manufacturing condition, and the predetermined material property, the first relationship being obtained based on the learning model,
a step of acquiring a second relationship that is the relationship obtained based on material simulation,
a step of determining presence or absence of similarity between the first relationship and the second relationship, and
a step of determining a need for relearning of the learning model based on a determination result of the presence or absence of the similarity.

9. A non-transitory computer readable medium storing a program for creating a learning model configured to predict a material property of a material based on a manufacturing condition, the program being intended to cause a computer to:
realize a verifying unit configured to verify the learning model for predicting, from a predetermined manufacturing condition including at least one condition, a predetermined material property of a material manufactured under the manufacturing condition, and
realize, as the verifying unit,
a first acquisition unit configured to acquire a first relationship that is a relationship between a selected condition, which is the condition selected from among the predetermined manufacturing condition, and the predetermined material property, the first relationship being obtained based on the learning model,
a second acquisition unit configured to acquire a second relationship that is the relationship obtained based on material simulation,
a similarity determination unit configured to determine presence or absence of similarity between the first relationship and the second relationship, and
a relearning determination unit configured to determine a need for relearning of the learning model based on a determination result of the presence or absence of the similarity.
